(19) United States
(12) Patent Application Publication
(10) Pub. No.: US 2025/0057744 A1
(43) Pub. Date: Feb. 20, 2025

(54) TWO-PHASE MAKEUP REMOVER WITH SURFACTANTS

(71) Applicant: BEIERSDORF AG, Hamburg (DE)

(72) Inventors: Saurabh GUPTA, Rajasthan (IN); Preksha LAAD, Madhya Pradesh (IN)

(21) Appl. No.: 18/719,371

(22) PCT Filed: Nov. 8, 2022
(86) PCT No.: PCT/EP2022/081125

§ 371 Date: Jun. 13, 2024

(30) Foreign Application Priority Data
Dec. 17, 2021 (IN) 202111058971

(51) Int. Cl. A61K 8/42; A61K 8/03; A61K 8/20; A61K 8/92

(52) U.S. Cl. CPC A61K 8/42; A61K 8/03; A61K 8/20; A61K 8/922; A61K 2800/30; A61K 2800/34

(57) ABSTRACT

The present invention belongs to the cosmetic field.

The present invention belongs to the cosmetic field and relates to a two-phase makeup remover.

A beautiful and attractive appearance is a desire for many people. One typical sign of such an appearance is a healthy and smooth looking skin. In order to take care on the skin, it is for many people a daily routine to apply decorative cosmetic products such as mascara or foundation. However, before going to bed, most people remove the decorative cosmetic products again. Conventionally the products are removed using a two-phase makeup remover, which has an aqueous phase and an oil phase, which are clearly separated.

The two-phase makeup remover is applied by shaking the product to mix both phases and then transferring some of the mixture onto a suitable tissue. The tissue is then used to cleanse and absorb the makeup from the skin.

The state of the art knows various two-phase makeup remover. Typical examples are described in U.S. Pat. No. 5,165,917A.

A typical ingredient in those products are silicone oils or silicone compounds as well as lower alkanes. Those compounds allow for an effective cleansing as well as a fast and effective phase separation after mixing the ingredients. That means no “sweating” is observed and the phases do not become hazy. The separation time should usually take 60 minutes.

In terms of the present disclosure the term “sweating” is understood as oil droplets clinging to the surface of bottle in water phase portion.

In terms of the present disclosure the term “hazy” is understood as not clear and transparent. A hazy formulation is not clear and transparent.

In terms of the present disclosure the term “lower alkanes” covers all alkanes, which do not have
a higher molecular weight than 480 Da,
a kinematic viscosity at 100° C. of >8.5 cSt, and
a carbon number at 5% boiling point of >25.

This definition is in line with the definition provided in COSMETICS EUROPE RECOMMENDATION N14 17-09-2018 “MINERAL HYDROCARBONS IN COSMETIC LIP CARE PRODUCTS”, available under the link https://www.cosmeticseurope.eu/files/3715/3907/8160/Recommendation_14_Mineral_Hydro_Carbons.pdf.

In terms of the present disclosure the term “silicone oils and silicone compounds” are understood as ingredients comprising a dimethylsiloxane unit.

However, the use of silicone oils and silicone compounds has been controversially discussed in recent years such that many consumers prefer to use products not containing those compounds.

Further the use of lower alkanes is limited by the cited COSMETICS EUROPE RECOMMENDATION N14 17-09-2018 in lip applications. In principle a conventional makeup remover is not used on the lips such that lower alkanes can be included. However, makeup remover may be used closed to sensitive areas such as the eyes and the lips such that it is generally desirable in the interest of the consumer to avoid using these compounds as well.

The absence of the silicone oils, silicone compounds and lower alkanes adversely affects the properties of two-phase makeup remover. Regarding the separation properties the separation time after mixing is often increased or extremely reduced. Further sweating effects are often observed. Further the separated phases become more translucent not allowing a clean and pretty appearance to be maintained over the use cycle of the product. Furthermore, the ability to remove waterproof makeup is generally found to be reduced.

Some of these effects are often addressed by adding nonionic surfactants. However, the use of surfactants in general should be limited to safe resources. Thus, it remains also desirable to provide products comprising limited amounts of surfactants, while showing no sweating and haziness 24 h after shaking, and reasonable separation times from 30 to 90 minutes.

Accordingly, it was the objective of the present invention to address, solve and/or reduce the shortfalls of the makeup removers containing no silicone oils and lower alkanes.

It was surprisingly found by the application that the objectives can be met by the present invention.

A first object of the present invention is a two-phase cosmetic cleansing composition, which is not an emulsion, comprising
a) An aqueous phase
b) An oil phase
   whereby the water phase comprises at least one amphoteric surfactant, preferably sodium coco amphoacetate and whereby the oil phase is free from silicone oils and lower alkanes.

Another object of the invention is the use of the composition according to the invention to remove makeup from the human skin.

It was surprisingly noticed that applying the subject matter of the invention sweating and separation time can be as requested, while maintaining low amounts of surfactants. Accordingly, the objectives of the present invention are solved.

All the weight percentages (% by weight) given below relate, unless otherwise stated, to the total weight of the cosmetic composition. If ratios of certain components are disclosed in the following description, these ratios refer, unless otherwise stated, to weight ratios of the components.

Unless otherwise stated, all tests and measurements were performed under “normal conditions”. The term “normal conditions” refers to 20° C., 1013 hPa and a relative humidity of 60%.

To define if an oil is a lower alkane, the following standardized methods are applied:

### Kinematic Viscosity at 100° C.:

ASTM D-445-21, published 15 May 2021

### Carbon Number at 5% Boiling Point:

The specification “carbon number≥25 at the 5% boiling point” means there is not more than 5% of hydrocarbons with a chain length less than 25. It can be determined by Gas Chromatography. A standard method is ASTM D 6352-19e1 published 1 Dec. 2019

### Molecular Weight:

The mean molecular weight can be determined from the kinematic viscosity of the oil. A standard protocol can be found in ASTM D2502-14(2019)e1 published 1 May 2019.

To define if a wax is a lower alkane, the following standardized methods are applied:

### Viscosity at 100° C.:

The same method as for oils (ASTM D-445) can also be used for waxes. If the material is not totally soluble in the normalized solvent of the method, the viscosity at 100° C. may be obtained by extrapolation from measurements performed at 120° C., 130° C. and 150° C.

### Carbon Number at 5% Boiling Point:

The specification “carbon number≥25 at the 5% boiling point” means there is not more than 5% of hydrocarbons with a chain length less than 25, which is consistent with food regulations. It can be determined by Gas Chromatography. Standard methods are ASTM D-2887 D2887-19ae2, published 1 Dec. 2019.

### Molecular Weight

The molecular weight is determined by osmometry in four different concentrations in toluene at 65° C.

In the following description the terms “according to the invention”, “preferred according to the invention” and so on are always directed to the cosmetic composition according to the invention, the use according to the invention and to the method according to the invention.

For the purposes of the present disclosure, the term “free from” means that the proportion of the respective substance is less than 0.05% by weight, preferably less than 0.01% by weight and most preferably 0% by weight, based on the total weight of the respective phase of the composition. Accordingly, the % by weight values for silicone oil and lower alkanes corresponds to the total weight of the oil phase. This definition ensures that entrainments or impurities with these substances are not included as “free from” according to the invention.

The term “skin” refers solely to the human skin.

Emulsifiers are understood to be all substances which are listed in the International Cosmetic Ingredient Dictionary and Handbook, Thirteenth Edition 2010, (ISBN 1-882621-47-6) under the name “emulsifying agent”. Surfactants are understood to be all substances which are listed in the International Cosmetic Ingredient Dictionary and Handbook, Thirteenth Edition 2010, (ISBN 1-882621-47-6) under the name “surfactant”.

It is preferred according to the invention if the ratio by volume between the aqueous phase and the oil phase is in the range from 20:1 to 1:20, preferably from 10:1 to 1:2, preferably 8:1 to 2:1, more preferably 7:1 to 3:1 and most preferably 6:1 to 4.5:1.

Further it is preferred if the aqueous phase comprises water in a total quantity of at least 60% by weight, more preferably at least 65% by weight, more preferably at least 70% by weight, more preferably at least 75% by weight, more preferably at least 80% by weight, more preferably at least 85% by weight and most preferably at least 90% by weight, calculated to the total weight of the aqueous phase.

Further it is preferred if the aqueous phase comprises water in a total quantity of less than 99% by weight, more preferably less than 98% by weight, more preferably less than 97% by weight, more preferably less than 96% by weight and most preferably less than 95.5% by weight, calculated to the total weight of the aqueous phase.

The aqueous phase comprises at least one amphoteric surfactant. Thereby, it is preferred if the total quantity of the amphoteric surfactants is in the range from 0.01 to 2% by weight, more preferably from 0.02 to 0.5% by weight, more preferably from 0.025 to 0.1% by weight and most preferably from 0.03 to 0.06% by weight, calculated to the total weight of the aqueous phase.

Preferred amphoteric surfactants can be selected from compounds of the following formulas (i) to (v), in which the group R in each case stands for a straight-chain or branched, saturated or mono- or polyunsaturated alkyl or alkenyl group having 8 to 24 carbon atoms,

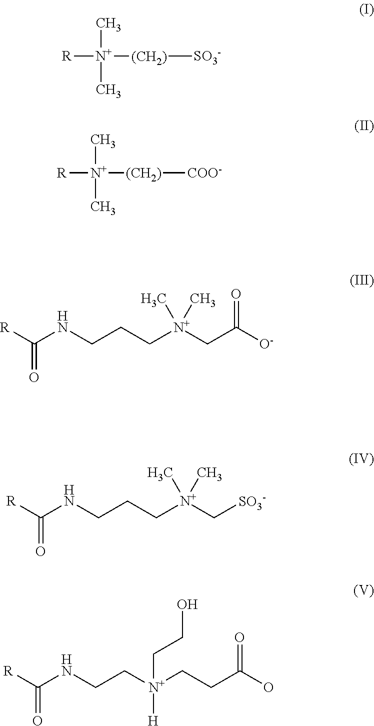

Especially suitable amphoteric surfactants are alkyl amidoalkyl betaines and/or alkyl ampho(di)acetates of the aforementioned formulas (i) to (v). Especially suitable amphoteric surfactants include the surfactants known under the INCI names Sodium Cocoamphoacetate, Cocamidopropyl Betaine and Disodium Cocoamphodiacetate. Most preferred is Sodium Cocoamphoacetate.

It is preferred if the total quantity of the Sodium Cocoamphoacetate is in the range from 0.01 to 2% by weight, more preferably from 0.02 to 0.5% by weight, more preferably from 0.025 to 0.1% by weight and most preferably from 0.03 to 0.06% by weight, calculated to the total weight of the aqueous phase. It was surprisingly noted that at such low levels of Sodium Cocoamphoacetate sweating effects were avoided and a separation time between 30 to 90 minutes was maintained. The phases were not hazy after 24 hours.

Further, it is preferred if the composition is free from anionic, nonionic and/or cationic surfactants.

According to the invention it is further preferred if the aqueous phase comprises at least on complex former, which is preferably EDTA.

Further it is preferred if the aqueous phase of the composition comprises at least one preservative. Preferred preservatives are selected from the group consisting of phenoxyethanol, ethylhexylglycerin and benzethonium chloride. Particular preferred are phenoxyethanol and/or benzethonium chloride.

For the case that phenoxyethanol is contained, it is preferred if the total quantity of phenoxyethanol is in the range from 0.1 to 1.5% by weight, more preferably 0.2 to 1.2% by weight and most preferably 0.4 to 0.8% by weight, calculated to the total weight of the aqueous phase.

For the case that benzethonium chloride is contained, it is preferred if the total quantity of benzethonium chloride is in the range from 0.01 to 0.5% by weight, more preferably 0.02 to 0.3% by weight and most preferably 0.04 to 0.2% by weight, calculated to the total weight of the aqueous phase.

A further typical ingredient preferably contained in the aqueous phase is glycerol. For the case that glycerol is contained, it is preferred if the total quantity of glycerol is in the range from 0.1 to 8.0% by weight, more preferably 0.5 to 6.0% by weight and most preferably 1.0 to 5.5% by weight, calculated to the total weight of the aqueous phase.

Further it is preferred if the aqueous phase comprises sodium chloride. Sodium chloride is preferably contained in quantities ranging from 0.01 to 0.6% by weight, more preferably 0.1 to 0.5% by weight and most preferably 0.15 to 0.35% by weight, calculated to the total weight of the aqueous phase.

The oil phase of the composition is preferably characterized in that
it has an interfacial tension towards water in the range from 22 to 35 mN/m,
a density in the range from 0.82 to 0.90 g/cm³, and/or
the oil phase is free from silicone oils and lower alkanes.

According to the invention the interfacial tension of the oil phase is determined using a Tensiometer K100 obtained from the Fa·Krüss.

The parameter for the measurement are described as follows:
Device: Tensiometer K100 (KRÜSS)
Correction: yes: Density
Temperature: 20° C.
Setting:
Measurement body: Krüss Standard Ring
Radius: 9.545 mm
Diameter of wire: 0.37 mm
Parameters:
Velocity Surface Detection: 4 mm/min
Sensitivity Surface—Detection: 0.004 g
Velocity Searching: 4 mm/min
Sensitivity Searching: 0.001 g
Velocity Measuring: 2 mm/min
Sensitivity Searching: 0.001 g
Immersion depth: 3.00 mm
Reversing distance: 10%
Values: 5-max. 20
Date acquisition: linear
The density of the oil phase is determined using a calculation method, which is outlined below:
For each individual oil contained in the formulation the density dᵢ was determined in the following manner:
The Density measurements were carried out under following conditions:
Method: Density (Triple determination)
Instrument: DMA 4500
Correction: yes: for Viscosity<700 mPas
System: flexural resonator
Temperature: 20° C.
To determine the density of the total oil phase D, the following calculation is performed:

$$D = 100 / ( {\sum }_{i}^{n} ( {x}_{i} / {d}_{i} ) )$$

Wherein xᵢ is the mass fraction of each individual oil i, and dᵢ is the density of the individual oil, wherein i is an integer ranging from 1 to n and n corresponds to the total number of individual oils in the oil phase.

Generally various oil compounds may be mixed. This can be seen by the example formulations, which comprise oil phases which are quite different, while these oil phases are optimized to have a specific interfacial tension and density as described above.

According to the invention some oils may preferably be contained in the formulations according to the invention.

Preferred oils according to the invention are ester oils, in particular ethylhexyl stearate, ethylhexyl cocoate, isodecyl neopentanoate, caprylic/capric triglyceride, cocoglycerides, isopropyl palmitate, dibutyl adipate, isoamyl laurate, isoamyl cocoate, propylene glycol dicaprylate/dicaprate, coco-caprylate/caprate, dicaprylyl carbonate, propylheptyl caprylate, decyl oleate, c12-15 alkyl benzoate and/or decyl cocoate.

Further it is preferred if natural oils are contained. Preferred natural oils are coconut oil, (sweet) almond oil, walnut oil, peach kernel oil, apricot kernel oil, avocado oil, tea tree oil, soybean oil, Glycine soja oil, sesame oil, sunflower oil, tsubaki oil, evening primrose oil, rice bran oil, palm kernel oil, mango kernel oil, cuckoo flower oil, thistle oil, macadamia nut oil, grape seed oil, amaranth seed oil, argan oil, bamboo oil, olive oil, wheat germ oil, pumpkin seed oil, mallow oil, hazelnut oil, safflower oil, canola oil, sasanqua oil, jojoba oil, rambutan oil, cocoa butter, vegetable oil and shea butter. Glycine soja oil and vegetable oil are preferred.

Further preferred oils are selected from ether oils, in particular dicaprylyl ether.

Further it is preferred if the quantity of oils that are liquid under normal condition is at least 80% by weight, more preferably 85% by weight, more preferably 90% by weight, more preferably 95% by weight, more preferably 98% by weight and most preferably at least 99% by weight, calculated to the total weight of the oil phase.

### EXAMPLES

The following examples should illustrate the compositions of this invention, without intending to limit the invention to these examples. The numerical values in the examples are percentages by weight, based on the total weight of each phase of the preparations.

The experimental results are indicated in the following table containing example formulations. All formulations designated with Coin. X are reference example not according to the invention, whereby X is an integer. All formulations designated with Ex. Y are examples according to the invention, whereby Y is an integer.

| | | | | Com. | Com. | Com. |
|---|---|---|---|---|---|---|
| | Ingredients | Ex.1 | Ex.2 | 1 | 2 | 3 |
| Water | Sodium chloride | 0.22 | 0.22 | 0.22 | 0.22 | 0.32 |
| phase: | Water | Ad | Ad | Ad | Ad | Ad |
| | | 100 | 100 | 100 | 100 | 100 |
| | Glycerin | 2.43 | 2.43 | 2.43 | 2.43 | 2.82 |
| | Benzethonium chloride | 0.06 | 0.06 | 0.06 | 0.06 | 0.06 |
| | Phenoxyethanol | 0.45 | 0.45 | 0.45 | 0.45 | 0.45 |
| | EDTA | 1.41 | 1.41 | 1.41 | 1.41 | 1.41 |
| | Sodium | 0.06 | 0.12 | | | |
| | Cocoamphoacetate | | | | | |
| | Coco Glucoside | | | | | |
| | Caprylyl/Capryl | | | 0.03 | | 0.06 |
| | Glucosie | | | | | |
| Oil | Isodecyl | | | | | |
| phase: | Neopentanoate | | | | | |
| | Caprylic/Capric | | | | | |
| | Triglyceride | | | | | |
| | Cocoglycerides | | | | | |
| | Isopropyl Palmitate | | | | | |
| | Glycine Soja Oil | | | | | |
| | Dibutyl Adipate | | | | | |
| | Vegetable Oil | | | | | |
| | Isoamyl Laurate | | | | | 34.08 |
| | Isoamyl Cocoate | | | | | |
| | Propylene Glycol | | | | | 22.21 |
| | Dicaprylate/Dicaprate | | | | | |
| | Coco-Caprylate/ | 1.48 | 1.48 | 1.48 | 1.48 | |
| | Caprate | | | | | |
| | Dicaprylyl Ether | 5.30 | 5.30 | 5.30 | 5.30 | |
| | Dicaprylyl Carbonate | | | | | |
| | Propylheptyl | Ad | Ad | Ad | Ad | |
| | Caprylate | 100 | 100 | 100 | 100 | |
| | Hydrogenated | 49.45 | 49.45 | 49.45 | 49.45 | Ad |
| | Polydecene | | | | | 100 |
| | Decyl Oleate | | | | | 4.47 |
| | Ethylhexyl Stearate | 11.09 | 11.09 | 11.09 | 11.09 | |
| | Ethylhexyl Cocoate | 9.22 | 9.22 | 9.22 | 9.22 | 10.16 |
| | C12-15 Alkyl | | | | | |
| | Benzoate | | | | | |
| | Decyl Cocoate | | | | | |
| Mix | Water phase | 85 | 85 | 85 | 85 | 85 |
| Quantity | Oil phase | 15 | 15 | 15 | 15 | 15 |
| (v/v) | | | | | | |
| Parameter | | | | | | |
| | Calculated Density | 0.84 | 0.84 | 0.84 | 0.84 | 0.86 |
| | (Oil Phase) g/cm3 | | | | | |
| | Interfacial Tension | 34.5 | 34.5 | 34.5 | 34.5 | 29.0 |
| | (Oil phase) mN/m | | | | | |
| Observation | | | | | | |
| (1 - yes, | Haziness at tsp | 1 | 1 | 1 | 1 | 1 |
| 0-No) | (binary) | | | | | |
| | Haziness at 24 | 0 | 0 | 1 | 0 | 0 |
| | H(binary) | | | | | |
| | Separation Time | 40 | 50 | 30 | 27 | 7 |
| | (tsp) (min) | | | | | |
| | Sweating: 24 | 0 | 0 | 0 | 1 | 0 |
| | H (binary) | | | | | |

The examples were analyzed in the following manner. After preparation of the samples the two-phase products were shake thoroughly for 10 second. Afterwards it was waited for a full separation of the phases (time stamp called tsp). The tsp. was noted. At this time point the haziness of the phases were analyzed by optical analysis. Further, 24 h after shaking the haziness of the phases and the appearance of sweating effect (oil droplets cling to plastic bottle surface in aqueous phase) was visually inspected. If a value was determined with a yes, the value 1 was added to the table above. For the case that no haziness was observed, or no sweating was observed a 0 was entered in the table.

As indicated by the table above superior results were obtained with the formulations according to the invention.

1.-10. (canceled)

11. A two-phase cosmetic cleansing composition, wherein the composition is not an emulsion and comprises
(a) an aqueous phase and
(b) an oil phase,
the aqueous phase comprising one or more amphoteric surfactants and the oil phase being free from silicone oils and lower alkanes.

12. The composition of claim 11, wherein the one or more amphoteric surfactants comprise sodium coco amphoacetate.

13. The composition of claim 11, wherein a ratio by volume between the aqueous phase and the oil phase ranges from 20:1 to 1:20.

14. The composition of claim 11, wherein a ratio by volume between the aqueous phase and the oil phase ranges from 10:1 to 1:2.

15. The composition of claim 11, wherein a ratio by volume between the aqueous phase and the oil phase ranges from 8:1 to 2:1.

16. The composition of claim 11, wherein a ratio by volume between the aqueous phase and the oil phase ranges from 7:1 to 3:1.

17. The composition of claim 11, wherein the aqueous phase comprises water in a total concentration of at least 60% by weight, based on a total weight of the water phase.

18. The composition of claim 11, wherein the aqueous phase comprises water in a total concentration of not more than 99% by weight, based on a total weight of the water phase.

19. The composition of claim 11, wherein a total concentration of the one or more amphoteric surfactants is from 0.01% to 2% by weight, based on a total weight of the aqueous phase.

20. The composition of claim 11, wherein a total concentration of the one or more amphoteric surfactants is from 0.02% to 0.5% by weight, based on a total weight of the aqueous phase.

21. The composition of claim 11, wherein a total concentration of the one or more amphoteric surfactants is from 0.025% to 0.1% by weight, based on a total weight of the aqueous phase.

22. The composition of claim 12, wherein the composition comprises from 0.01% to 2% by weight of sodium coco amphoacetate, based on a total weight of the aqueous phase.

23. The composition of claim 12, wherein the composition comprises from 0.02% to 0.5% by weight of sodium coco amphoacetate, based on a total weight of the aqueous phase.

24. The composition of claim 12, wherein the composition comprises from 0.025% to 0.1% by weight of sodium coco amphoacetate, based on a total weight of the aqueous phase.

25. The composition of claim 11, wherein the aqueous phase comprises sodium chloride.

26. The composition of claim 25, wherein the sodium chloride is present in a concentration of from 0.01% to 0.6% by weight, based on a total weight of the aqueous phase.

27. The composition of claim 25, wherein the sodium chloride is present in a concentration of from 0.1% to 0.5% by weight, based on a total weight of the aqueous phase.

28. The composition of claim 11, wherein the oil phase has an interfacial tension towards water of from 22 mN/m to 35 mN/m.

29. The composition of claim 11, wherein the oil phase has a density of from 0.82 g/cm³ to 0.90 g/cm³.

30. The composition of claim 28, wherein the oil phase has a density of from 0.82 g/cm³ to 0.90 g/cm³.
